(19) United States
(12) Patent Application Publication
(10) Pub. No.: US 2026/0363533 A1
(43) Pub. Date: Nov. 19, 2026

(54) METHOD AND SYSTEM FOR CONTROLLING AN ORAL CAVITY PHOTOGRAPHY

(71) Applicant: Guangzhou Stars Pulse co., Ltd., Guangzhou (CN)

(72) Inventors: Hang Fan, Guangzhou (CN); Bo Chen, Guangzhou (CN); Hanhong Dai, Guangzhou (CN)

(73) Assignee: Guangzhou Stars Pulse co., Ltd., Guangzhou (CN)

(21) Appl. No.: 19/206,464

(22) Filed: May 13, 2025

(51) Int. Cl. A61B 1/045; A61B 1/00; A61B 1/06; A61B 1/24; A61B 5/00; H04N 23/10; H04N 23/60; H04N 23/667; H04N 23/74

(52) U.S. Cl. CPC A61B 1/045; A61B 1/00045; A61B 1/0638; A61B 1/0655; A61B 1/24; A61B 5/0071; A61B 5/0088; H04N 23/10; H04N 23/64; H04N 23/667; H04N 23/74

(57) ABSTRACT

The present disclosure provides systems for an oral cavity photography, and methods controlling therefor. The method comprises: obtaining photographic setup specification of the oral cavity photography system, wherein the photographic setup specification is directly or indirectly corresponded to a distance between the camera module and a subject to be photographed; and determining a target parameter based on the photographic setup specification, wherein the target parameter includes at least one of a light-emitting parameter for controlling the light-emitting module to illuminate the subject and an imaging parameter for controlling the camera module to capture an image.

### BACKGROUND

The disclosed subject matter, in some embodiments thereof, relates to oral photography techniques, and more particularly, to a method, device, and system for controlling oral cavity photography across at least one photographic area within the oral cavity.

Photography and digital imaging have been integrated to dentistry for both diagnostic and cosmetic purposes, including by illuminating an intra-oral region with visible or invisible light to capture photographic, reflectance, or fluorescence images. To acquire high-quality images, supplementary or filling light may be used to enhance visibility of the subject being examined. However, variations in photographic conditions associated with a subject may result in degraded image quality with overexposure or insufficient illumination. This inconsistency may impede the detection of subtle dental anomalies, which can be important for accurate oral diagnosis and effective treatment.

For example, while the exposure of images in certain areas, such as the upper occlusal surface, might be balanced, others like the lower occlusal surface or the outer side of the incisor may be overexposed with the same imaging parameters. This inconsistency can pose challenges in capturing uniformly high-quality images across all intra-oral regions, potentially leading to diagnostic inaccuracies or necessitating repeated imaging sessions.

Therefore, there is a need to develop a reliable method for an oral cavity photography.

### SUMMARY

The disclosed subject matter enhances diagnostic capabilities by providing more uniform and reliable visual image quality. The disclosed subject matter provides techniques for controlling an oral cavity photography to ensure consistent image quality across different areas of the oral cavity, through adjusting at least one of relating photographic parameters.

One aspect of the disclosed subject matter provides a method for controlling an oral cavity photography system, comprising a camera module and a light-emitting module. The method includes obtaining a photographic setup specification of the oral cavity photography system, wherein the photographic setup specification directly or indirectly corresponds to a distance between the camera module and a subject to be photographed, and the camera module comprises a camera with a Field of View (FOV) greater than 140°; and determining a target parameter based on the photographic setup specification. The target parameter includes at least one of a light-emitting parameter for controlling the light-emitting module to illuminate the subject and an imaging parameter for controlling the camera to capture an image. In some embodiments, the method further comprises adjusting one or more of the light-emitting parameter and the imaging parameter based on the target parameter.

In some embodiments, the photographic setup specification is determined by at least one of the following: images captured by the camera, distance data from a distance sensor, an orientation of the camera in the oral cavity, and a position of the camera in the oral cavity. The photographic setup specification corresponds to at least one of the following: an oral region corresponding to the subject and the area thereof, a distance between the subject and the camera module, a photography position of the camera module within the oral cavity, and an entrance state of the camera module, wherein the entrance state of the camera module includes an intraoral state or an extraoral state. In some embodiments, the oral region comprises a tooth position and/or a tooth surface, and at least two oral regions correspond to different target parameters, respectively; and/or the photography position of the camera module within the oral cavity includes tooth position information and/or tooth surface information, and at least two photography positions correspond to different target parameters, respectively.

In some embodiments, the light-emitting parameter comprises at least one of the following: a target power of the light-emitting module, a target brightness of the light-emitting module, or a target number of illuminated lights in the light-emitting module; and the imaging parameter comprises at least one of the following: a target exposure duration of the camera module, a target exposure gain of the camera module, or a target sensitivity of the camera module; and the target parameter has a positive correlation with the distance or area of the oral region. The extraoral state and the intraoral state correspond to different target parameters, respectively.

In some embodiments, the positive correlation between the target parameter and the distance comprises multiple distance intervals, each corresponding to different target parameters, and a first distance interval among the multiple distance intervals corresponds to a smaller target parameter than that of a second distance interval, wherein a maximum value of the first distance interval is less than or equal to a minimum value of the second distance interval. The light-emitting parameter of the target parameter corresponding to the intraoral state is greater than that corresponding to the extraoral state. In some embodiments, the oral cavity photography system further comprises a detachable mouth opener, and when the mouth opener is in a connected state, the entrance state is an extraoral state; and when the mouth opener is in a detached state, the entrance state is an intraoral state.

In some embodiments, the method further comprises adjusting the image after capturing through enhancing a visualization of the image or enhancing a display of a region of interest (ROI).

In some embodiments, adjusting the target parameter comprises a fixed value adjustment in which the light-emitting parameters or imaging parameters are adjusted to preset values or ranges, or comprises a variable value adjustment in which the light-emitting parameters or imaging parameters are adjusted in real-time. In some embodiments, adjusting one or more of the light-emitting parameters and the imaging parameters comprises simultaneously adjusting the light-emitting parameters and the imaging parameters.

In some embodiments, the method further comprises outputting or saving the image, wherein the image comprises a panoramic image of an occlusal surface of teeth.

In some embodiments, adjusting one or more of the light-emitting parameter and the imaging parameter comprises operating in a first photography mode and/or a second photography mode, wherein in the first photography mode, the light-emitting module emits light in a first wavelength band configured for illumination; in the second photography mode, the light-emitting module emits at least light in a second wavelength band configured to excite plaque fluorescence for display; and the target parameters determined based on the photographic setup specification are at least partially different between the first photography mode and the second photography mode.

In some embodiments, in the first photography mode, the imaging parameter of the target parameter is determined based on the photographic setup specification, and in the second photography mode, both the light-emitting parameter and the imaging parameter of the target parameter are determined based on the photographic setup specification.

In some embodiments, in the second photography mode, the light-emitting module further emits light in the first wavelength band, and when determining and adjusting the lighting parameters of the target parameter based on the photographic setup specification, the adjustment values for the light in the first wavelength band and the second wavelength band are different.

In some embodiments, the first wavelength band corresponds to white light with a wavelength of 380-750 nm, and the second wavelength band corresponds to violet light with a wavelength of 380-450 nm or blue light with a wavelength of 450-500 nm.

Another aspect of the disclosed subject matter provides an oral cavity photography system comprises a processor, an oral scanner connected to the processor, the scanner comprising a camera with a sensor having a Field of View (FOV) greater than 140°, and a light-emitting module; and a non-transitory computer-readable medium coupled to the processor and comprising instructions for performing the method described above. The camera module comprises an oral insertion with the camera and the emitting unit thereon. The oral scanner comprises an opener configured to surround the outer edge of the camera.

Another aspect of the disclosed subject matter provides a method for controlling light for a photographic area of an oral cavity photography comprises identifying a photographic setup specification of the photographic area by a camera with a sensor within the oral cavity, through a judgment of the sensor on preset data saved thereon, and adjusting light-emitting parameters based on the identified photographic area, thereby maintaining the sensor to receive substantial light inlet parameters.

### DESCRIPTION OF THE DRAWINGS

FIG. 1 is a schematic diagram illustrating a system for oral cavity photography, according to an embodiment of the disclosed subject matter.

FIGS. 2A-2D illustrate exemplary different photographic areas in the oral cavity, according to an embodiment of the disclosed subject matter.

FIG. 3A-3B is flowchart of an adjustment for one or more of light-emitting parameters and imaging parameters, according to an embodiment of the disclosed subject matter.

FIG. 4 illustrate a gray scale image with weighted average value for brightness of captured images, according to an embodiment of the disclosed subject matter.

FIG. 5 illustrates a flowchart for an adjustment by applying a PID control, according to an embodiment of the disclosed subject matter.

FIG. 6A-6B illustrates a structure for a camera module with or without an opener, according to an embodiment of the disclosed subject matter.

### DETAILED DESCRIPTION

The disclosed technology improves the quality of oral cavity photographs, making captured images clearer and more consistent by using techniques that adjust certain photographic parameters to ensure uniform image quality across all areas in the mouth.

The terms used in the disclosure generally have their ordinary meanings in the art, within the context of the subject matter and in the specific context where each term is used. The term “light-emitting parameter(s)” refers to the settings or characteristics of the light source, such as brightness, intensity, color temperature, etc. The term “imaging parameter(s)” refers to settings of a camera related to capturing the image itself, like focus, contrast, exposure, and shutter speed. Herein, the light-emitting parameter and imaging parameter can be collectively referred to as “scanning parameter”. The term “illumination parameter(s)” refers to settings detected a sensor receiving the light source. The term “light inlet parameter(s)” refers to settings or characteristics how the light enters the sensor or camera, such as the aperture size, the angle of light entering, the size of the sensor, and the shutter speed. The light inlet parameter can be detected by a sensor, and can be determined via adjusting at least one of the light-emitting parameter or imaging parameter and can affect how clear and detailed the final image appears in the subject matter. The term “image parameter(s)” refers to visual characteristics of captured images, such as the brightness, exposure uniformity of the captured images. The image parameter defines the quality and appearance of the capture images. The term “photographic area” or “photographic region” refers to the specific portion of the oral cavity that is captured by the camera module of the oral cavity photography system. The term “target parameter” refers to a parameter including a (target) light-emitting parameter and/or a (target) imaging parameter.

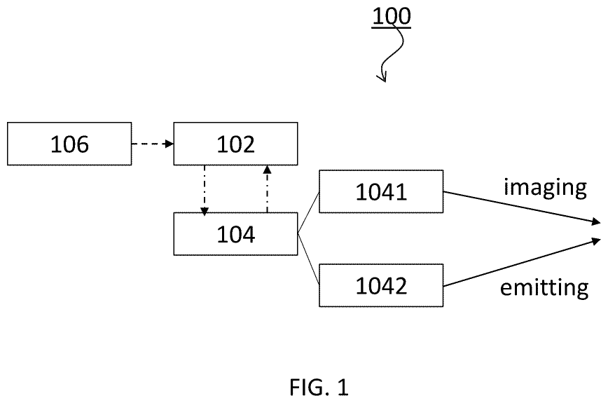

With reference to FIG. 1, the oral cavity photography system 100 includes an oral imaging unit 104 having a light-emitting module 1041 and a camera module 1042. As further discussed below, the light-emitting module 1041 and the camera module 1042 are operated in accordance with certain target parameters, including at least one of light-emitting parameters and/or imaging parameters. In accordance with the disclosed subject matter, the system can make suitable adjustments to such the parameters to enhance image quality.

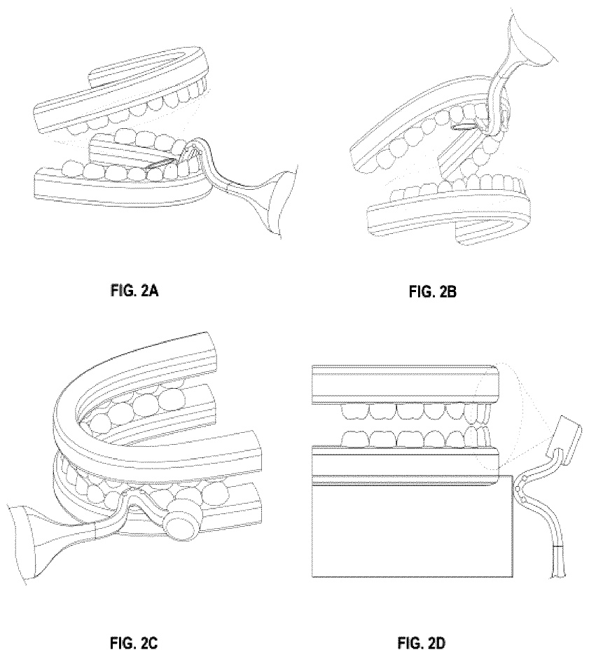

As shown in FIGS. 2A-2D, photographic areas may comprise an upper/lower occlusal surface (FIG. 2A, FIG. 2B), left/right outer side surface (FIG. 2C), and/or the outer side of the incisor (FIG. 2D). In some embodiments, image quality may be quantitatively measured using at least one of exposure uniformity and brightness of the captured images. In some embodiments, a captured image is considered as having good quality if: (1) oral anomalies present in the captured images, regardless of photographic areas, are apparent and recognizable to the naked eye; (2) visual features or attributes, such as image exposure, are consistent across different photographic areas of the oral cavity; (3) the image color is neutrally balanced, or the impact of a colored light source is minimized; and (4) captured images meet the requirements for dentistry clinical documentation.

The system 100 may comprise at least a processor 102, a memory 106, and an oral imaging unit 104 (e.g., an oral scanner). The oral imaging unit 104 further includes a light-emitting module 1041 and a camera module 1042. The processor 102 may be a programmable logic device, a microcontroller, a microprocessor, or any suitable combination of the preceding. The processor 102 is not limited to a single processing device and may encompass multiple processing devices. The processor 102 may operate to process information, control the system 100, and perform any of the functions described herein. The processor 102 is communicatively coupled to and in signal communication with the memory 103. The memory 106 may include a volatile memory (e.g., RAM, ROM, or DRAM) for temporary data storage and non-volatile memory (e.g., flash storage or a hard drive) for long-term data retention. The memory 106 is operable (or configured) to store information used by the processor 102 and/or any other logic and/or instructions for performing the functions described in this disclosure.

The system 100 may comprise instructions for performing oral cavity photography for capturing images of at least one area within the oral cavity. Such photography may be implemented through a combination of hardware and software components installed on the memory 103 or a non-transitory computer-readable medium (not depicted). The hardware may further include an input/output (I/O) interface for communication with other system components for operating the adjustments for various parameters, and data storage device for storing photographic data, imaging results, and computational algorithms. The software component includes algorithms that enable the system to receive, save, and process various parameters. These algorithms may include (Region of Interests) ROI model or machine learning techniques to calculate the brightness of the light in the oral cavity more accurately.

In some embodiments, the light-emitting module 1041 includes at least one emitting component, configured to emit light at one or more wavelengths, within both the visible and invisible spectra. The emitting unit may comprise one or more light-emitting components, such as LEDs, which can be arranged in different patterns, such as dots, rings, or spheres. For example, a plurality of light-emitting components may be arranged around the camera 1042 in a ring shape.

In some embodiments, the light-emitting unit 1041 may comprise components capable of emitting ultraviolet, white, warm white, blue, purple, or red light etc. In some embodiments, ultraviolet light may be used to stimulate the fluorescent display of dental plaques. In another embodiment, white light may be used to compensate for the white balance or fill light. A person of ordinary skill would understand that a selection of color or color combination may be determined based on the particular use case of the current invention. In some embodiments, the light-emitting module 1041 is operated to illuminate at least one photographic area of the oral cavity under the control of light-emitting parameters. In some embodiments, the light-emitting parameters can include but are not limited to at least one of light-emitting brightness, light power, light illuminance, light intensity, and luminous flux. In some embodiments, the color or color combination may also be part of the light-emitting parameters.

In some embodiments, the camera module 1042 may comprise a camera, which may be a compact digital camera, a macro camera, a wide-angle camera, and/or a 360-degree camera. In some embodiments, the camera may have a Field of View (FOV) greater than 140°, covering a large photographic area, such as the occlusal surface. The camera module 1042 may be operated to capture images of one or more photographic areas under the control of imaging parameters. In some embodiments, the imaging parameters can include at least one of camera sensitivity (ISO), shutter speed, aperture, exposure compensation, white balance, metering mode, light color, and focus mode. The camera module 1042 may further comprise a light sensor for calibrating supplemental light.

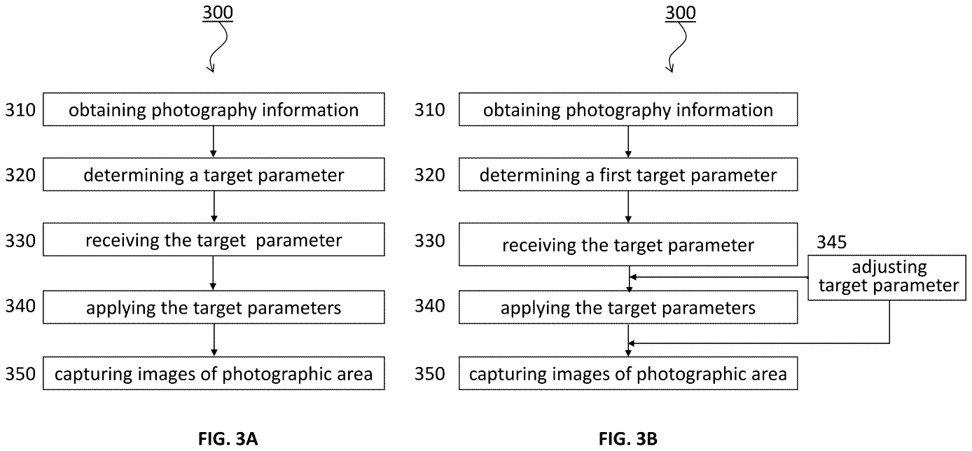

With reference to FIG. 3A, the disclosed subject matter provides an exemplary method for controlling an oral cavity photography. At 310, a photographic setup specification of the system 100 is obtained, which may include subject distance, subjection location, camera location, shooting area, and camera status. At 320, one or more target parameters may be determined based on the photographic setup specification. The target parameters may include at least one of a light-emitting parameter for controlling the light-emitting module to illuminate the subject and an imaging parameter for controlling the camera module to capture an image. The target parameter(s) may further include exposure settings, which may include aperture, exposure time, shutter speed, exposure gain, ISO settings, and the like. At 330, a camera or a sensor within the system 100 is positioned in the oral cavity to receive the target parameter corresponding to the photographic setup specification obtained above. At 340, the system operates the oral scanner to apply the determined target parameter to actuate the shutter of camera for capturing. In particular, various target parameters are predetermined based on the photographic setup specification and stored in the system, securing a graded adjustment mode (will be discussed later). At 350, images of the photographic area in the oral cavity are captured.

The photographic setup specification is significantly correlated to the photographic area within the oral cavity. In some embodiments, the photographic setup specification can be corresponded to at least one of the following: the images captured by the camera, data from a distance sensor, an orientation of the camera in the oral cavity, a position of the camera in the oral cavity. In addition, the photographic setup specification is determined least one of the following: an oral region corresponding to the subject and area thereof, a distance between the subject and the camera module, a photography position of the camera module within the oral cavity, and an entrance state of the camera module; wherein the entrance state of the camera module comprising an intraoral state or an extraoral state. The extraoral state and the intraoral state correspond to different target parameters, respectively, and a light-emitting parameter of the target parameter corresponding to the intraoral state is greater than that corresponding to the extraoral state. The oral region comprises a tooth position and/or a tooth surface, and at least two oral regions correspond to different target parameters, respectively; and/or the photography position of the camera module within the oral cavity includes a tooth position and/or a tooth surface, and at least two photography positions correspond to different target parameters, respectively.

For example, when capturing the occlusal surface in the intraoral state, if the entire occlusal surface needs to be imaged while the camera's focal length remains fixed, the camera needs to be positioned farther away from the occlusal surface, requiring larger target parameters (e.g., higher brightness or power). In contrast, when capturing the labial/buccal surface of teeth in the extraoral state—such as when dividing the surface into multiple images (e.g., three) for separate shots—the camera is positioned relatively closer to the surface, thus requiring smaller target parameters (e.g., lower brightness or power). Furthermore, each photographic setup specification, e.g., distance and/or position of the camera, may correspond to a different target parameter. For instance, photographing the upper molar's occlusal surface may require a different combination of exposure gain and LED brightness than capturing the labial surface of the lower incisor. In practice, the system may maintain a predefine schedule or mapping function that links specific photographic setup specifications to target parameters in a graded adjustment mode, enabling real-time or near real-time adjustment during the controlling. In some embodiments, the system can store historical usage data, so that once a particular photographic area is identified (e.g., anterior maxillary region), the system can apply preset target parameters corresponding to prior successful imaging conditions, further enhancing accuracy and reducing image variability.

As previously noted, different photographic setup specification corresponds to distinct target parameters. For instance, first photographic setup specification, such as a first distance, corresponds to a first target parameter; second photographic setup specification, such as a second distance, corresponds to a second target parameter; and so on. This enables the system to implement a controllable graded adjustment method.

In some embodiments, the system 100 can determine the initial value of light-emitting parameters, which may be used to control the light-emitting module 1041. In some embodiments, system 100 may store one or more sets of initial light-emitting parameters, each of which may correspond to one or more photographic setup specifications. The initial parameters of photographic setup specification are configured to create substantially uniform illumination for the camera module 1042. In some embodiments, the initial light-emitting parameter may be manually adjusted by a user. In some embodiments, the initial light-emitting parameters may be dynamically determined based on photography conditions. At 320, the light-emitting module 1041 emits light in accordance with the light-emitting parameters. Light-emitting operation may be controlled with techniques known to the art, such as voltage control, current control, Pulse Width Modulation, etc.

In some embodiments, the initial light-emitting parameters for illuminating during the photography can be preset parameters corresponding to the photographic setup specification, while the imaging parameters are dynamically adjusted based on some circumstances. The preset parameters corresponding to a photographic setup specification may be configured to attain substantially consistent illumination conditions. In some embodiments, the target parameters can be represented by Image Signal Processor (ISP) configurations, each of which corresponds to one or more photographic areas.

In some embodiments, the simultaneous adjustment comprises both adjusting the light-emitting parameters and adjusting the imaging parameters to obtain a light inlet parameter, thereby capturing images. The adjusting the target parameter comprises a manual adjustment through identifying different preset adjustment models for different photographic setup specification. Alternatively, the adjust the target parameter comprises an automatic adjustment by a controller.

In some embodiments, the light-emitting parameter comprises at least one of the following: a target power of the light-emitting module, a target brightness of the light-emitting module, a target number of illuminated lights in the light-emitting module; the imaging parameter includes at least one of the following: a target exposure duration of the camera module, a target exposure gain of the camera module, a target sensitivity of the camera module. The target parameter has a positively correlation with the distance and area of the oral region. The positive correlation between the target parameter and the distance comprises multiple distance intervals, each corresponding to different target parameters, and a first distance interval among the multiple distance intervals corresponds to a smaller target parameter than that of a second distance interval, and the upper limit of a first distance interval is less than or equal to the lower limit of a second distance interval.

In some embodiments, the target parameter is positively correlated with the distance between the camera module and the oral region being imaged, and/or the area of the region. This positive correlation enables adaptive control where more distant or larger photographic regions require stronger illumination or longer exposure. For example, photographing the lower occlusal surface may require greater brightness and exposure time compared to imaging the anterior teeth due to differences in accessibility and light diffusion. To avoid continuous recalculation and provide real-time responsiveness, this correlation is implemented as a stepwise or graded adjustment based on discrete distance intervals. Each interval corresponds to a distinct set of target parameters. For example:

A first distance interval (e.g., 0-10 mm) may correspond to a lower brightness setting (e.g., 70%);

A second distance interval (e.g., 11-20 mm) may correspond to higher brightness (e.g., 100%).

The maximum value of the first interval is less than or equal to the minimum value of the second interval, ensuring non-overlapping adjustment bands. In this way, the target parameter remains constant within each defined interval but grades up when the system detects that the distance to the subject has entered the next interval. This discretization enables the system to quickly retrieve pre-calibrated parameter sets, rather than calculating values continuously. For instance, if the system detects that the camera is imaging a larger occlusal surface from a farther distance (e.g., 15 mm), it may select a high-power lighting setting (e.g., 100% brightness with the white light activated) and increase the camera's sensitivity, while imaging for the inner side of incisors (e.g., 5 mm), the system may select a medium lighting setting (e.g., 70% brightness, fewer white light) and a lower exposure gain to avoid overexposure. That is, the target parameter does not change continuously with distance, but rather has one value within a certain distance range and a larger value in a farther distance range.

In some embodiments where the system operates flexible adjustments until the target parameter is obtained, the sensor is designed to obtain a light inlet parameter, e.g., light inlet brightness or luminousness, detected by the sensor, after initial light-emitting in the oral. Based on the obtained light inlet parameter, the system is operated to adjust one or more of the light-emitting parameters and the imaging parameters, securing the target parameter corresponding to the target image. Notably, the light inlet parameters are detected or observed parameters corresponding to the illumination conditions in which the camera is located. The parameters may comprise illuminance, light intensity, luminous flux, uniformity, etc. The light inlet parameters may not be substantially consistent across different photographic areas of the oral cavity, e.g., having an extensive exposure difference for different photographic areas of the subject, thereby failing to complete a precision diagnosis for oral cavity. In such embodiments, the system 100 may continuously adjust the light-emitting parameters or imaging parameters until the exposure uniformity of the captured images is reached. In some embodiments, one or more light-emitting parameters and imaging parameters may be adjusted to account for variations of the light inlet parameters of the camera, which may contribute to the image quality of the captured images. One advantage of adjusting the light-emitting parameters and imaging parameters is to attain substantially consistent illumination conditions across various photographic areas in the oral cavity, thereby enhancing uniformity of exposure in the captured images. For example, one of formulas for calculating the light inlet parameter is:

Light Inlet=F×S×L+t

Where F represents the aperture size, S represents the sensor size, L represents the light-emitting intensity, one of light-emitting parameters, t represents the shutter speed, one of imaging parameters.

The adjustable factors are L and t. Therefore, the light inlet parameter can be regulated through at least one of the light-emitting parameters of the light-emitting unit or imaging parameters of the camera, thereby enhancing the image parameter or image quality of the captured images.

As referenced in FIG. 3B, at 345, the system may adjust target parameters in multiple times. In some embodiments where the light inlet parameters for photographic areas are different after merely adjusting an target parameter during the method, the sensor control the processor to continuously adjust the target parameters until the light inlet parameters are satisfied to be consistence cross various photographic areas, thereby capture an image having an uniform brightness.

In some embodiments, the system 10 can operate in various photography modes, e.g., via graded adjustments. In the first exemplary photography mode, the light-emitting module emits light in a first wavelength band, wherein the first wavelength band is configured for illumination; in the second exemplary photography mode, the light-emitting module emits at least light in a second wavelength band, wherein the second wavelength band is configured to excite plaque fluorescence for display; the target parameters determined based on the photographic setup specification are at least partially different between the first photography mode and the second photography mode. In the first photography mode, the imaging parameters of the target parameter are determined based on the photographic setup specification, and in the second photography mode, both the light-emitting parameter and the imaging parameter of the target parameter are determined based on the photographic setup specification. In further embodiments, in the second photography mode, the light-emitting module further emits light in the first wavelength band, and when determining and adjusting the lighting parameters of the target parameter based on the photographic setup specification, the adjustment values for the light in the first wavelength band and the second wavelength band are different. The first wavelength band corresponds to white light, and the second wavelength band corresponds to violet light or blue light.

Table 1 listing some specific parameters associated violet light and white light for different photographic areas for the above photography modes.

TABLE 1
Violet Light (first exemplary photography mode)

| | | | | | |
|---|---|---|---|---|---|
| Violet | White Light | White | Auto Exposure | White | |
| Light | Supplementary | Light | Expected Mean | Balance | |
| Brightness | Brightness | Brightness | (Exposure Gain) | Multipliers | Brightness & Contrast |

Upper Occlusal Surface

| | | | | | |
|---|---|---|---|---|---|
| 100% | 50% | 0% | 1000 | R: 1.5625 | Y Component Gain: 280 |
| | | | | G: 1 | U Component Gain: 600 |
| | | | | B: 1.757 | V Component Gain: 600 |

Lower Occlusal Surface

| | | | | | |
|---|---|---|---|---|---|
| 100% | 50% | 0% | 960 | R: 1.5625 | Y Component Gain: 280 |
| | | | | G: 1 | U Component Gain: 600 |
| | | | | B: 1.757 | V Component Gain: 600 |

Anterior Teeth (Outer Side)

| | | | | | |
|---|---|---|---|---|---|
| 70% | 10% | 0% | 1500 | R: 1.5625 | Y Component Gain: 256 |
| | | | | G: 1 | U Component Gain: 800 |
| | | | | B: 1.757 | V Component Gain: 800 |

Left/Right Outer Side

| | | | | | |
|---|---|---|---|---|---|
| 80% | 10% | 0% | 1500 | R: 1.5625 | Y Component Gain: 256 |
| | | | | G: 1 | U Component Gain: 800 |
| | | | | B: 1.757 | V Component Gain: 800 |

Left/Right Inner Side

| | | | | | |
|---|---|---|---|---|---|
| 100% | 50% | 0% | 1500 | R: 1.5625 | Y Component Gain: 280 |
| | | | | G: 1 | U Component Gain: 600 |
| | | | | B: 1.757 | V Component Gain: 600 |

White Light (second exemplary photography mode)

| | | | | | |
|---|---|---|---|---|---|
| Violet | White Light | White | | White | |
| Light | Supplementary | Light | Auto Exposure | Balance | |
| Brightness | Brightness | Brightness | Expected Mean | Multipliers | Brightness & Contrast |

Upper Occlusal Surface

| | | | | | |
|---|---|---|---|---|---|
| 0% | 100% | 10% | 1000 | R: 1.3671 | Y Component Gain: 280 |
| | | | | G: 1 | U Component Gain: 600 |
| | | | | B: 1.757 | V Component Gain: 600 |

Lower Occlusal Surface

| | | | | | |
|---|---|---|---|---|---|
| 0% | 100% | 10% | 788 | R: 1.3671 | Y Component Gain: 280 |
| | | | | G: 1 | U Component Gain: 600 |
| | | | | B: 1.757 | V Component Gain: 600 |

Anterior Teeth (Outer Side)

| | | | | | |
|---|---|---|---|---|---|
| 0% | 100% | 10% | 960 | R: 1.3671 | Y Component Gain: 280 |
| | | | | G: 1 | U Component Gain: 600 |
| | | | | B: 1.757 | V Component Gain: 600 |

Left/Right Outer Side

| | | | | | |
|---|---|---|---|---|---|
| 0% | 100% | 10% | 1500 | R: 1.3671 | Y Component Gain: 280 |
| | | | | G: 1 | U Component Gain: 600 |
| | | | | B: 1.757 | V Component Gain: 600 |

Left/Right Inner Side

| | | | | | |
|---|---|---|---|---|---|
| 0% | 100% | 10% | 2500 | R: 1.3671 | Y Component Gain: 280 |
| | | | | G: 1 | U Component Gain: 600 |
| | | | | B: 1.757 | V Component Gain: 600 |

Table 1 illustrates certain parameters data sources for the graded photography mode as noted before. Based on Table 1, the oral cavity photography system dynamically adjusts its imaging parameters (Auto Exposure Expected Mean) and light-emitting parameters (e.g., Light Brightness, White Light Supplementary Brightness White, Light Brightness) depending on the selected photography mode and the photographic area within the oral cavity, so that certain substantial same target parameters, e.g., White Balance Multipliers and Brightness&Contrast, can be obtained. Table 1 illustrates specific target parameters, including imaging parameters and light-emitting parameters, for various intraoral regions (e.g., occlusal surfaces, anterior and lateral tooth surfaces). In the first photography mode, white light (380-750 nm) is used primarily for illumination, while in the second mode, violet light (380-450 nm), or an alternative of blue light (450-500 nm), is used to induce plaque fluorescence. Notably, in the second mode, both violet and white light may be used simultaneously, but their respective brightness and exposure settings differ across oral regions. For instance, occlusal surfaces tend to use higher violet light brightness (100%) with adjusted gain and exposure to enhance plaque visibility, while anterior or lateral surfaces may operate with balanced or reduced brightness to maintain contrast. Table 1 has demonstrated how the system fine-tunes target parameters per region and mode for controlling the oral cavity photography, supporting tailored diagnostics and image clarity under different lighting conditions.

Additionally, an identification of visual features of the captured images, including image brightness and exposure uniformity, can be used to determine if the target parameter needs an adjustment. Through an analysis on the captured images, and at least one adjustment of light-emitting parameters and imaging parameters can ensure that the images are adjusted to meet the corresponding target parameters. Alternatively, an energy receiving unit can be added to the oral imaging unit 104, and the light-emitting parameters and imaging parameters can be adjusted accordingly based on the received energy. Herein, the image parameters can be represented by exposure uniformity or brightness of the captured images.

The target parameters may be managed to maintain uniform exposure and brightness, e.g., the same weighted average value of exposure or light brightness for multiple divided regions in a gray scale image, for captured images of different photographic areas in the oral cavity. Specifically, at least one of the light-emitting parameter and the imaging parameter are adjusted until the target parameters are substantially consistent across different photographic setup specification that corresponds to different photographic areas, respectively. The light-emitting parameters and imaging parameters have preset parameters (e.g., values or ranges), corresponding to individual photographic areas, which determine the adjustment extension of image parameters and/or light-emitting parameters. Notably, the preset parameters can be used to obtain substantially consistent parameters or values, or illumination parameters to the sensor of the camera, regardless of differences photographic areas. For example, an upper occlusal surface as the photographic area is corresponded to a light-emitting parameter(s) “E1”, which can be a specific type of light-emitting parameters, e.g., light brightness 100%, or a combination for more than two parameters, e.g., light brightness 100% and auto exposure target mean value 1000. Comparatively, left/right side incisor as the photographic area is corresponded to a specific light-emitting parameter(s) “E2”, featured as, light brightness 70%, or a combination for light brightness 70% and auto exposure target mean value 1500. When operating a photography in the above two different areas of oral cavity, an input of parameters “E1” and “E2” respectively, allows to output a substantially the same light inlet parameter or target parameter, creating substantially the same image quality of two captured images, e.g., uniform exposure or brightness for two captured images. If the light inlet parameters or target parameters are not substantially the same for different photographic areas, an additional adjustment of the imaging parameters can be made until the desired consistency is achieved. Thus, for such adjustments, the imaging parameters are further adjusted adaptively based on the light inlet parameter or target parameter, while the light-emitting parameters are adjusted based on a preset pattern.

Light-emitting parameters may account for different characteristics of individual photographic areas including location (e.g., occlusal surface versus outer side incisor), distance (long versus short), size (larger versus smaller areas), and detection patterns (e.g., intraoral versus extraoral detection). Generally, longer distances and larger areas require higher values of some light-emitting parameters, e.g., brightness, power, and illuminance. In some embodiments, the light-emitting parameters include the color or color combinations of supplemental light. In some cases, the color of the captured image may drift from normal when non-white lights such as blue and purple lights are irradiated into the mouth. When only colored light is used, the overall color of the image may deviate from neutral. By adding white light for supplementary lighting, the overall white balance of the image can be adjusted more efficiently.

In some embodiments, preset default values may be assigned to the light-emitting parameters based on the individual photographic areas. In some embodiments, the parameters may be further adaptively adjusted to account for conditions of individual photographic areas. By starting the adjustment with preset values for a particular photographic area, rapid and precise adjustments may be achieved.

In some embodiments, target parameters may be utilized for better parameter adjustments. Examples of target parameters may include visual attributes of captured images, such as brightness, color, exposure intensity, etc. Target parameters may be a range of acceptable values, a single value, or a mix of both. Ideally, when the visual attributes of captured images fall within or meet the target parameters, the observed light inlet parameters or obtained target parameters also fall within a preset range accordingly.

In some embodiments, the operation for applying different light-emitting parameters for adjusting comprises at least one of the following ways: adjusting the power of individual light-emitting components, controlling the number of active ones, and/or selecting the color or color combinations. Herein, when the light-emitting module 1041 comprises more than one color, the relative composition of colors may remain the same when being adjusted. In some embodiments, light-emitting components may comprise functional and filling ones. The functional light-emitting components are the ones used to stimulate certain areas for a diagnosis, while filling lamp is used for illuminating or white balance compensation. In some embodiments, the extent of amplitude of functional ones may be greater than that of the filling ones. Ideally, there should be no dark areas in the middle and edges, or overexposure.

In some embodiments, the captured images may be adjusted based on a region of interest (ROI). For example, an enhanced adjustment may be applied to an identified ROI, increasing the readability of the images without changing other regions. For example, when the occlusal surface is suitable for photography, ROI can be set to “U” type to ignore the oral tongue part, accurately calculate the brightness of the teeth, and then control the brightness of the light by a Proportional-Integral-Derivative controller (PID).

In some embodiments, the light-emitting parameters and/or imaging parameters can be automatically adjusted or manually selected to adjust a corresponding preset model, for various photographic areas. Automatically, the light-emitting parameters may be determined from the preset values or ranges corresponding to photographic areas through the preset. Alternatively, the oral scanning unit 104 is pre-programmed with multiple adjustment models for automatic or manual adjustments of the light-emitting parameters. The photographic area may be either automatically or manually selected.

In some embodiments, the oral imaging unit 104 can capture a panoramic image of the occlusal surface of the teeth, comprising the occlusal surface, inner side surface and outer side surface. Optionally, the oral imaging unit 104 has a structure installed thereof for securing the oral imaging unit, stabilizing the photographic distance and photographic areas, and maintaining consistency of light inlet and target parameters.

In some embodiments, light brightness of the emitted light may be controlled to make the light inlet parameters or target parameters of the sensor maintain substantially consistent and fall within a preset range for different photographic areas in the oral cavity. In some embodiments, the light brightness control can include at least one of the following operation: (1) adjusting the light brightness based on various photographic areas, e.g., upper/bottom occlusal surface versus side surface of the teeth; (2) adjusting the light brightness of the emitted light in real time according to brightness of the captured image; (3) adjusting the light brightness according to the distance from the light-emitting unit to the subject, wherein different color light, e.g., warm white light and ultraviolet light, are considered into the adjustment.

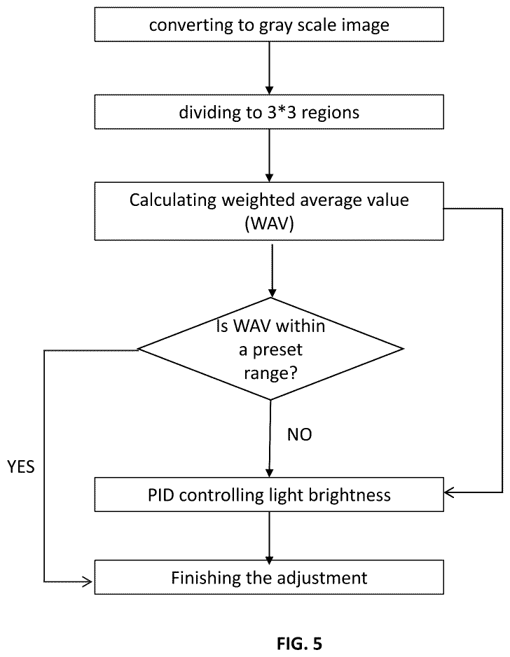

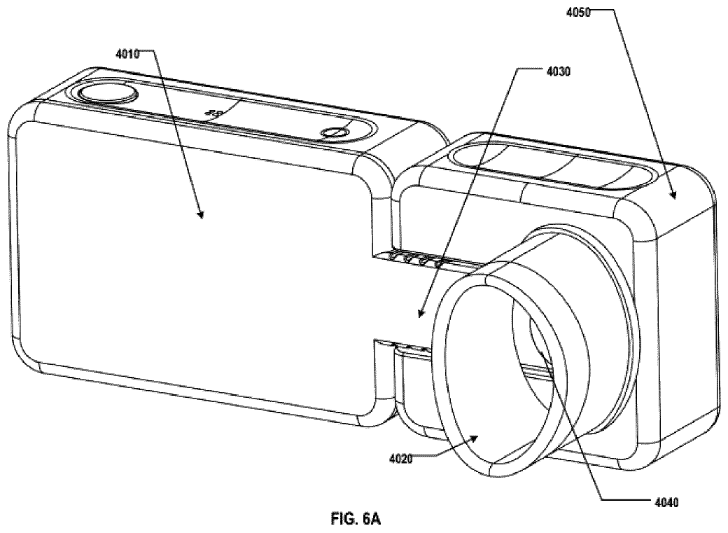

FIG. 5 shows an exemplary flowchart of adjusting scanner parameters. In some embodiments, when operating the adjustment, the oral cavity can be divided into a number of photographic areas: upper occlusal surface, lower occlusal surface, medial and side incisors, left/right side surface, left/right medial, etc. The system 100 may identify the photographic area being examined and apply light-emitting parameters, such as light brightness, correspondingly. In some embodiments, the photographic area may be identified using one or more following methods: (1) use the artificial intelligence (AI) model trained for oral cavity areas classification to automatically identify the current photographic areas through the captured image; (2) use a three/six axis sensor to distinguish various photographic areas due to differences in the angle and orientation of cameras in these various areas; and (3) manually set the photographic areas, where the light brightness is preset in the respective area.

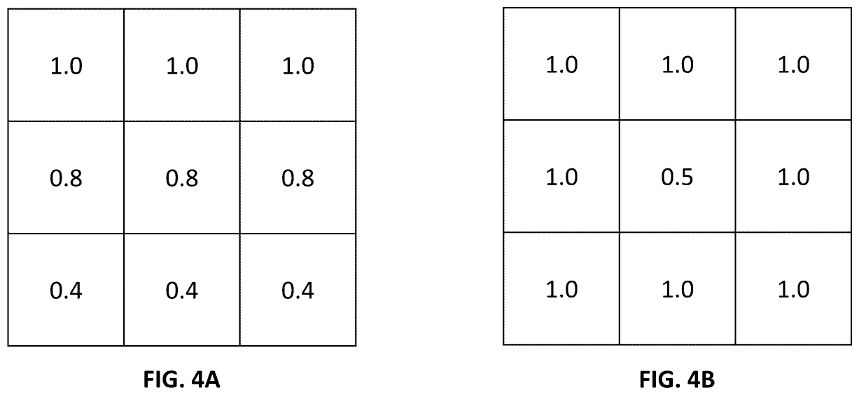

In some embodiments, light-emitting parameters, such as brightness, maybe determined using (a) different weighting matrices or (b) Region of Interests (ROI). To implement the different weighting matrices, the captured image may be converted into a gray scale image and divided into multiple regions (e.g., 3×3 regions), each of which may be assigned with a weight. FIGS. 4A and 4B show two exemplary weight metrices. For example, FIG. 4A shows an exemplary matrix for upper and lower occlusal surfaces, in which case, the pre-adjusted brightness distribution is typically dark in the upper half and bright in the lower half. FIG. 4B shows an exemplary matrix for cheek and lip imaging, in which case, the pre-adjusted brightness distribution is typically bright in the middle part and dark in the surrounding part.

In some embodiments, a controller, e.g., Proportional-Integral-Derivative (PID), is used to adjust and control the light-emitting parameters, such as light brightness, to maintain the captured image quality. An exemplary operation flow for the above adjustment is shown as in FIG. 5. The weighted average value of light brightness can also be adjusted by adjusting the exposure gain and exposure time. In addition to PID, the control instruction can also comprise a fuzzy control, adaptive control, optimal control, machine learning and other algorithms. In alternative embodiments, ROI can be set to calculate the light brightness in real time, so that when the current photographic area is identified, different ROI can be established to calculate the brightness of the subject more accurately. For example, when the occlusal surface is subject to be photographed, ROI can be setup efficient to “U” type, to ignore insignificant parts, e.g., the oral tongue part, thereby accurately calculating the required light brightness for photographing the teeth, and then controlling the brightness of the emitted light by PID.

In some embodiments, an infrared distance sensor can be installed on the same surface of the camera module to adjusting light brightness of the emitted light according to the distance from the light-emitting unit to the subject. By measuring the distance between the lens of the camera and the subject, adjusting the light brightness of the emitted light in real time can be controlled by PID, based on the preset distance range saved on the processor. In addition to infrared distance sensing, ultrasonic sensing, laser sensing, and image visual sensing can also be used to obtain the distance from the camera to the photographed subject.

Alternatively or additionally, ISP parameters of the sensor can be configured for various photographic areas to attain a uniformity of the captured image. An exemplary method with ISP configurations includes configuring the preset ISP parameters for various photographic areas according to the standard dental card; then, identifying various photographic areas including each of different photographic setup specification, respectively, by areas recognition described above; finally, applying preset ISP parameters corresponding to the photographic areas in a real time photographing, so that the captured images for different photographic areas is maintained to be substantially consistent. In some embodiments, the ISP parameters include sharpening, brightness contrast, color temperature, expected mean value of automatic exposure, automatic exposure compensation value, gamma curve, brightness, saturation and white balance, etc. The ISP parameters are preset and saved in the memory of the system.

In some embodiments, a photographic area may be identified using captured images, the position sensor, and/or proximity sensor. For example, the position sensor may be a multi axis sensor, or a visual sensor/photoelectric sensor integrated to the oral imaging unit 104. The proximity sensor may identify proximity or distance using ultrasound, photelectric, laser, optical, or a combination of the above.

Additionally or alternatively, the photographic areas can be identified by peripheral devices. For example, a detachable mouth opener may be used in combination with the scanning unit 103 when the outer side of the teeth is being examined while the mouth opener may be removed when occlusal surface is being examined. In addition, a sensor may be integrated to the mouth opener to obtain light inlet parameters. When the mouth opener is in a connected state, the entrance state is an extraoral state; when the mouth opener is in a detached state, the entrance state is an intraoral state.

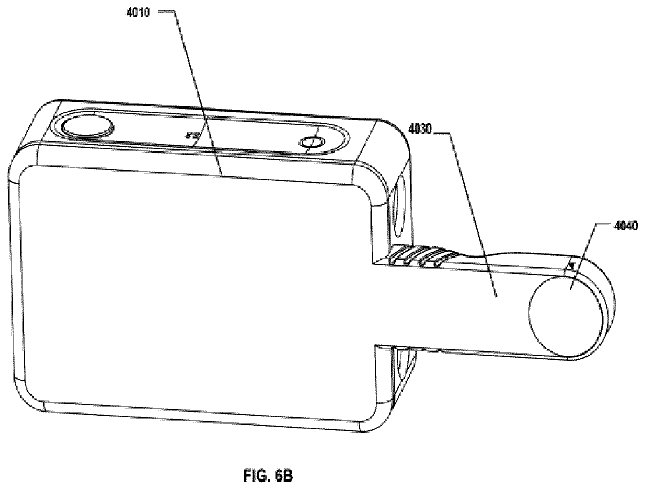

FIG. 6A illustrates an exemplary camera module 4000 with a mouth opener typically used for incisor outer surface imaging, which may comprise a main body 4010, a camera 4040 with a sensor (not shown), a mouth opener 4020 surrounding the outer edge of the camera 4040, a housing 4050 attached to the main body 4010 on a surface thereof, and an insertion 4030 extending from the main body 4010 to the housing 4050. In such example, the main body 4010 serves as the base structure, engaging other components and providing stability. The camera 4040 captures high-quality images of the incisor outer surface. The mouth opener 4020 is installed to secure the photographic position and orientation. The housing 4030 can support and secure other components. The insertion 4030 can provide a connection and structural integrity to the whole camera module 4000. Alternatively, FIG. 6B illustrates an exemplary camera module with a mouth opener removed, comprising a main body 4010, an insertion 4030 extending outward from the main body 4010, and a camera attached to the insertion 4030.

In addition to the detailed embodiments depicted above and claimed, the disclosed subject matter is also directed to other embodiments having other combinations of the features disclosed and claimed herein. As such, the features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

What is claimed is:

1. A method for controlling an oral cavity photography system, comprising a camera module and a light-emitting module, the method comprising:
obtaining a photographic setup specification of the oral cavity photography system, wherein the photographic setup specification directly or indirectly corresponds to a distance between the camera module and a subject to be photographed; and
determining a target parameter based on the photographic setup specification, wherein the target parameter includes at least one of a light-emitting parameter for controlling the light-emitting module to illuminate the subject and an imaging parameter for controlling the camera module to capture an image.

2. The method of claim 1, further comprises adjusting one or more of adjusting the light-emitting parameter and adjusting the imaging parameter based on the target parameter.

3. The method of claim 1, wherein the photographic setup specification is determined by at least one of the following: the images captured by the camera module, distance data from a distance sensor, an orientation of the camera module in the oral cavity, and a position of the camera module in the oral cavity.

4. The method of claim 1, wherein the photographic setup specification is corresponded to at least one of the following information: an oral region corresponding to the subject and area thereof, a distance between the subject and the camera module, a photography position of the camera module within the oral cavity, and an entrance state of the camera module; wherein the entrance state of the camera module comprising an intraoral state or an extraoral state.

5. The method according to claim 4, wherein the oral region comprises a tooth position and/or a tooth surface, and at least two oral regions correspond to different target parameters, respectively; and/or,
the photography position of the camera module within the oral cavity includes a tooth position information and/or a tooth surface information, and at least two photography positions correspond to different target parameters, respectively.

6. The method according to claim 4, wherein the light-emitting parameter comprises at least one of the following: a target power of the light-emitting module, a target brightness of the light-emitting module, a target number of illuminated lights in the light-emitting module; wherein the imaging parameter comprises at least one of the following: a target exposure duration of the camera module, a target exposure gain of the camera module, a target sensitivity of the camera module; wherein the target parameter has a positively correlation with the distance or area of the oral region.

7. The method according to claim 6, wherein the positive correlation between the target parameter and the distance comprises multiple distance intervals, each corresponding to each of different target parameters, and a first distance interval among the multiple distance intervals corresponds to a smaller target parameter than that of a second distance interval; wherein a maximum value of a first distance interval is less than or equal to a minimum value of a second distance interval.

8. The method according to claim 4, wherein the extraoral state and the intraoral state correspond to different target parameters, respectively.

9. The method according to claim 8, wherein the oral cavity photography system further comprises a detachable mouth opener, and when the mouth opener is in a connected state, the entrance state is an extraoral state; when the mouth opener is in a detached state, the entrance state is an intraoral state.

10. The method according to claim 1, further comprises adjusting the image after capturing through enhancing a visualization of the image or enhancing a display of a region of interest (ROI).

11. The method according to claim 1, wherein the adjusting the target parameter comprises a fixed value adjustment where the light-emitting parameters or imaging parameters are adjusted to preset values or ranges, or comprises a variable value adjustment where the light-emitting parameters or imaging parameters are adjusted in real-time.

12. The method according to claim 1, wherein the adjusting one or more of the light-emitting parameters and the imaging parameters comprises a simultaneous adjustment on the light-emitting parameters and imaging parameters.

13. The method according to claim 1, further comprising outputting or saving the image, wherein the image comprises a panoramic image of an occlusal surface of teeth.

14. The method according to claim 2, wherein the adjusting one or more of adjusting the light-emitting parameter and adjusting the imaging parameter comprises operating in a first photography mode and/or a second photography mode; wherein in the first photography mode, the light-emitting module emits light in a first wavelength band, wherein the first wavelength band is configured for illumination; in the second photography mode, the light-emitting module emits at least light in a second wavelength band, wherein the second wavelength band is configured to excite plaque fluorescence for display; the target parameter determined based on the photographic setup specification are at least partially different between the first photography mode and the second photography mode.

15. The method according to claim 14, wherein in the first photography mode, the imaging parameter of the target parameter are determined based on the photographic setup specification, and in the second photography mode, both the light-emitting parameter and the imaging parameter of the target parameter are determined based on the photographic setup specification.

16. The method according to claim 14, wherein, in the second photography mode, the light-emitting module further emits light in the first wavelength band, and when determining and adjusting the lighting parameters of the target parameters based on the photographic setup specification, the adjustment values for the light in the first wavelength band and the second wavelength band are different.

17. The method according to claim 16, wherein the first wavelength band corresponds to white light with a wavelength at 380-750 nm, and the second wavelength band corresponds to violet light with a wavelength at 380-450 nm or blue light with a wavelength at 450-500 nm.

18. An oral cavity photography system, comprising:
a processor,
an oral scanner connected to the processor, comprising:
a camera module, wherein comprises a Field of View (FOV) greater than 140°, and
a light-emitting module, and
a non-transitory computer-readable medium, coupled to the processor and comprising instructions for performing a method according to claim 1.

19. The system of claim 18, wherein the oral scanner comprises an opener configured to surround the outer edge of the camera.

20. A method for controlling light for a photographic area of an oral cavity photography, comprising:
identifying photographic setup specification of the photographic area by a camera with a sensor within the oral cavity, through a judgement of the sensor on preset data saved thereon, and
adjusting light-emitting parameters based on the identified photographic area, thereby maintaining the sensor to receive substantial light inlet parameters.
